(19) United States
(12) Patent Application Publication
(10) Pub. No.: US 2026/0000077 A1
(43) Pub. Date: Jan. 1, 2026

(54) CANNALACTONE ANALOGS, SYNTHESIS AND USE FOR STIMULATION OF GERMINATION OF PARASITIC PLANT SEEDS

(71) Applicants: CENTRE NATIONAL DE LA RECHERCHE SCIENTIFIQUE (CNRS), PARIS (FR); NANTES UNIVERSITE, NANTES Cedex 1 (FR); HEMP IT ADN, BEAUFORT-EN-ANJOU (FR)

(72) Inventors: François Didier BOYER, Le Chesnay-Rocquencourt (FR); Jean Bernard POUVREAU, Nort sur Erdre (FR); Suzanne DAIGNAN FORNIER DE LACHAUX, Massy (FR); Fabienne MATHIS, Angers (FR)

(21) Appl. No.: 19/256,849

(22) Filed: Jul. 1, 2025

(30) Foreign Application Priority Data
Jul. 1, 2024 (FR) FR2407160

(51) Int. Cl. A01N 43/08; A01P 13/02; C07D 405/12

(52) U.S. Cl. CPC A01N 43/08; A01P 13/02; C07D 405/12

(57) ABSTRACT

The present invention relates to the chemical synthesis of cannalactone analogs, as well as the use of these analogs for the stimulation of the germination of parasitic plant seeds.

### TECHNICAL FIELD OF THE INVENTION

The present invention relates to the chemical synthesis of cannalactone analogs, as well as the use of these analogs to stimulate the germination of parasitic plant seeds.

### TECHNICAL BACKGROUND

Hemp (Cannabis sativa) is an annual plant originating in Asia and has been used for more than 8,000 years. It is cultivated around the world and is able to cover the four vital needs of humanity: food, housing, clothing, medicine.

Hemp cultivation is widespread because it is a profitable and sustainable production. Indeed, the plant adapts easily to different soils and climates and as such does not require any phytosanitary treatment. The hemp production area in France has increased by a factor of thirty between 1960 and today. This interest in hemp is only increasing, partly with the aim of gradually replacing cotton, which is very water-intensive. France is today the leading hemp producer in Europe with a production of around 20,000 ha.

Known for its resistance to parasites and pests, the hemp plant is all the more attractive. However, a parasitic plant in the Orobanche family, the branched orobanche, Phelipanche ramosa induces large yield losses on hemp crops of up to more than 808. This parasite also attacks crops such as rapeseed or tobacco, but a specialization of a population of hemp branched orobanche has been brought to light [1]-[4].

After germination, the parasitic plant connects to the root of the host plant and thus recovers nutrients for its own development [5]. This parasitism creates significant damage to hemp crops, which can even lead to the abandonment of this crop on the field.

Strigolactones are small molecules known to be exuded in the soil at picomolar concentrations (10⁻¹² M) and have been identified as germination stimulants of Phelipanche ramosa or branched orobanche seeds [6], [7]. Strigolactones (SL) were first identified for their role in parasitic [8] and symbiotic [9] interactions in the rhizosphere and are the last class of plant hormones to be discovered [10], [11]. They are best known for their role in controlling plant architecture, more recently, roles for SLs in other aspects of plant development have been brought to light.

In order to have bioactive molecules more easily, synthetic analogs of strigolactones have been developed [4], [7], [13], [14]. Analogs are molecules with a structure similar to that of SLs but which do not potentially exist in Nature.

Following the discovery of cannalactone, the strigolactone found in hemp exudates in very small quantities and which is the main germination stimulant of P. ramosa [1]-[4], the Applicant has developed analogs that are easier to access compared to cannalactone and have a biological activity in some cases greater than the biological activity of cannalactone.

### SUMMARY OF THE INVENTION

In particular, the present invention relates to an analog of cannalactone, characterized in that it corresponds to the general Formula 1:

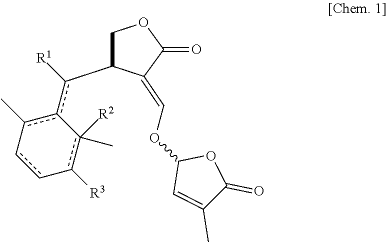

wherein:
   R¹ denotes the hydrogen atom H, the hydroxyl group OH or the OSiR⁴₃ group,
   R² and R³ each denote the hydrogen atom H or the methyl radical CH₃,
   R⁴ denotes an alkyl group, and
   the 6-membered carbon ring which may be aromatic or of the cyclohexene or cyclohexane type.

According to a first embodiment of the invention, the cannalactone analog according to the invention may be aromatic of “cis” and “trans” stereochemistry and corresponding to Formula 2:

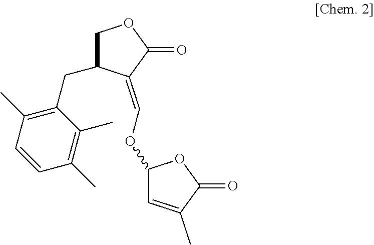

wherein:
   R¹, R² and R³ denote the hydrogen atom H, and
   the 6-membered carbon ring is aromatic.

According to a second embodiment of the invention, the cannalactone analog according to the invention may be of the “cis” and “trans” stereochemistry diene type and corresponds to Formula 3:

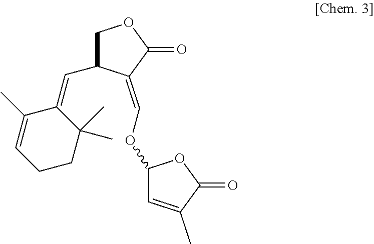

wherein:
   R¹ and R³ denote the hydrogen atom H,
   R² denotes the methyl group, and
   the 6-membered carbon ring is of the cyclohexene type.

According to a third embodiment of the invention, the cannalactone analog according to the invention may be of the “cis” and “trans” silylated stereochemistry type and corresponds to Formula 4:

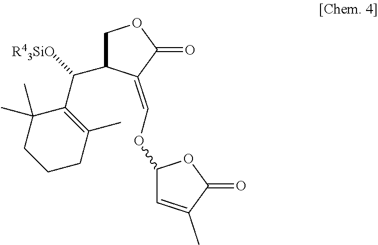

wherein:
   R¹ denotes the group OSiR⁴₃,
   R² denotes the methyl group,
   R³ denotes the hydrogen atom H, and
   the 6-membered carbon ring is of the cyclohexene type.

According to a fourth embodiment of the invention, the cannalactone analog according to the invention may be of the “cis” and “trans” stereochemical alcohol type and corresponds to Formula 5:

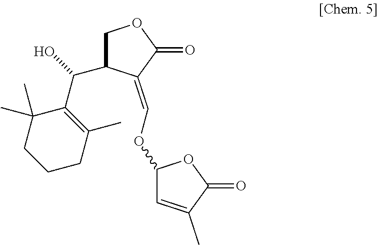

wherein:
   R¹ denotes hydroxyl group OH,
   R² denotes the methyl group,
   R³ denotes the hydrogen atom H, and
   the 6-membered carbon ring is of the cyclohexene type.

The present invention also relates to a method for synthesizing an analog of cannalactone according to the first embodiment, characterized in that it comprises the following steps:
a reaction A) of coupling commercial β-cyclocitral with a bromofuran C4 of Formula 6:

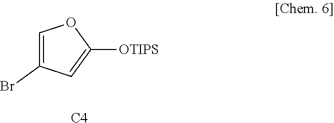

to obtain an alcohol B20 of Formula 7:

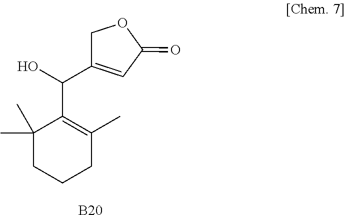

a step B) of reducing the alcohol B20 of Formula 7, to obtain a mixture of diastereoisomers of the allyl alcohol, followed by a step of separating said diastereoisomers to retain the diastereoisomer (4R*, 6R*)-B21 of Formula 8:

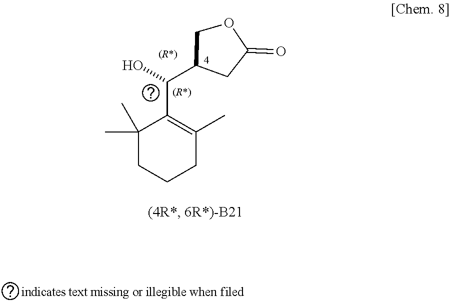

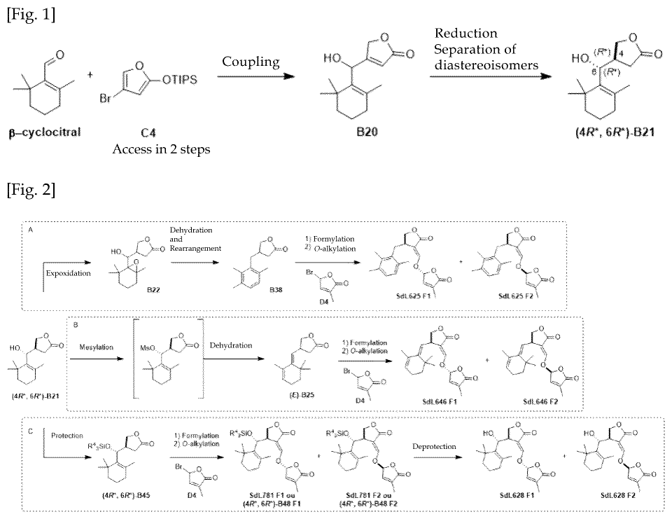

This synthesis is shown in [FIG. 1].
a step C2) of epoxidizing the diastereoisomer (4R*, 6R*)-B21 of Formula 8 to obtain an epoxy alcohol B22 of Formula 9:

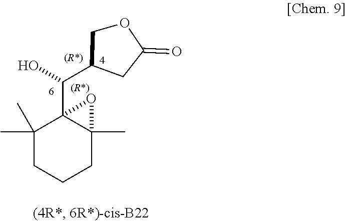

a step D2) of dehydrating and rearranging the epoxy alcohol B22 of Formula 9 to obtain a benzyl compound B38 of Formula 10:

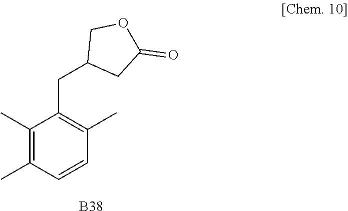 [SMILES: "B38" ; Cc1ccc(C)c(CC2COC(=O)C2)c1C]

a step E2) of formylation in basic medium with an alkyl formate of the benzyl compound B38 of Formula 10 to obtain an enol B40 of Formula 11:

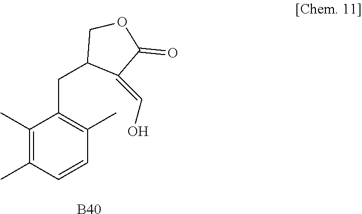 [SMILES: "B40" ; Cc1ccc(C)c(CC2COC(=O)/C2=C/O)c1C]

a step F2) of O-alkylation of enol B40 to obtain the analog of Formula 2 (of the aromatic type).

The alkyl formate (in particular an ethyl or methyl formate) corresponds to Formula 12

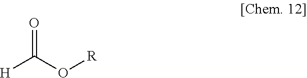

This synthesis is shown in [FIG. 2] (part A).

The present invention also relates to a method for synthesizing an analog of cannalactone according to the second embodiment, characterized in that it comprises the following steps:
steps A and B as defined in the method for synthesizing an analog of cannalactone according to the first embodiment, followed by
a step C3) of mesylating the diastereoisomer (4R*, 6R*)-B21 of Formula 8 to obtain after dehydration and rearrangement the diene (E)-B25 of Formula 13:

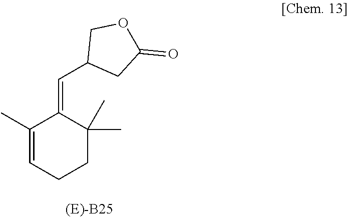 [SMILES: CC1=CCCC(C)(C)/C1=C\C1COC(=O)C1]

a step E3) of formylation in basic medium of diene (E)-B25 of Formula 13 to obtain an enol B42 of Formula 14:

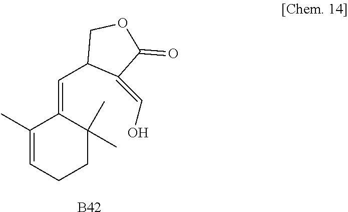 [SMILES: "B42" ; CC1=CCCC(C)(C)/C1=C\C1COC(=O)/C1=C/O]

then
a step F3) of O-alkylation of enol B42 to obtain the analog of Formula 3 (diene type).

This synthesis is shown in [FIG. 2] (part B).

The present invention also relates to a method for synthesizing an analog of cannalactone according to the third embodiment, characterized in that it comprises the following steps:
steps A and B as defined in the method for synthesizing a cannalactone analog according to the first embodiment, followed by
a step C4) of protecting the diastereoisomer (4R*, 6R*)-B21 of Formula 8 to obtain the protected compound (4R*, 6R*)-B45 of Formula 15:

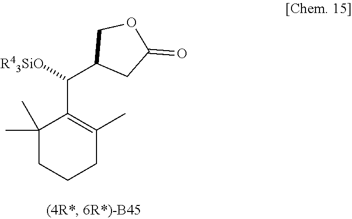

a step E4) of formylation in basic medium of the protected compound of Formula 14, to obtain an enol (4R*, 6R*)-B46 of Formula 16:

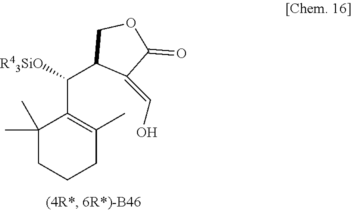

a step F4) of O-alkylation of enol B46 to obtain the analog of Formula 4 (of the silyl type).

This synthesis is shown in [FIG. 2] (part C).

The present invention also relates to a method for synthesizing an analog of cannalactone according to the fourth embodiment, characterized in that it comprises the following steps:
steps A and B as defined in the method for synthesizing a cannalactone analog according to the first embodiment, followed by
forming a cannalactone analog according to the third embodiment, followed by
a step G5) of deprotecting and separating the diastereoisomers of the analog of Formula 4, to obtain the analog of Formula 5 (alcohol type).

This synthesis is also shown in [FIG. 2] (part C).

The present invention further relates to the use of a cannalactone analog according to the invention or as obtained according to one of the methods for synthesizing according to the invention, as a germination stimulant of parasitic plant seeds.

In particular, it may be used as a germination stimulant of P. ramosa 1 and P. ramosa 2a seeds, or for the
suicidal germination of parasitic plants of the Striga, Orobanche and Phelipanche type.

### BRIEF DESCRIPTION OF THE FIGURES

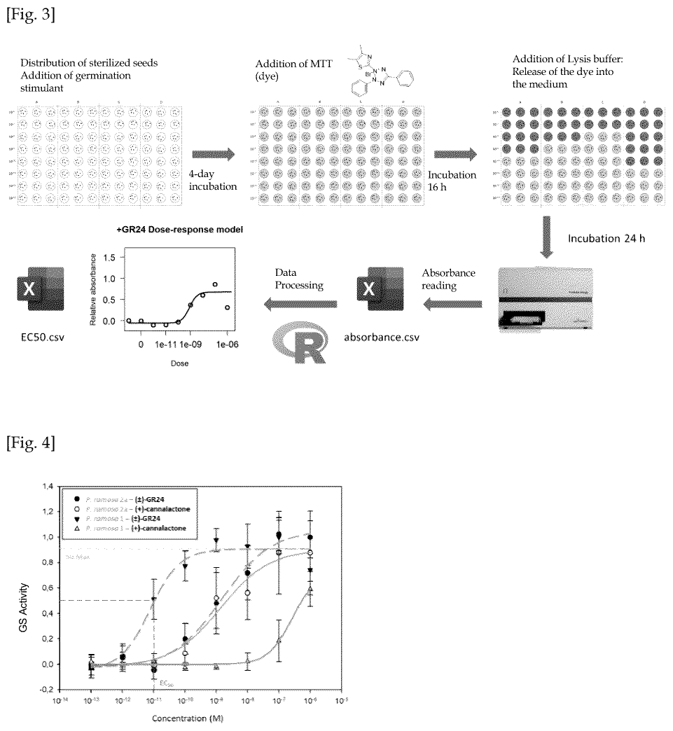

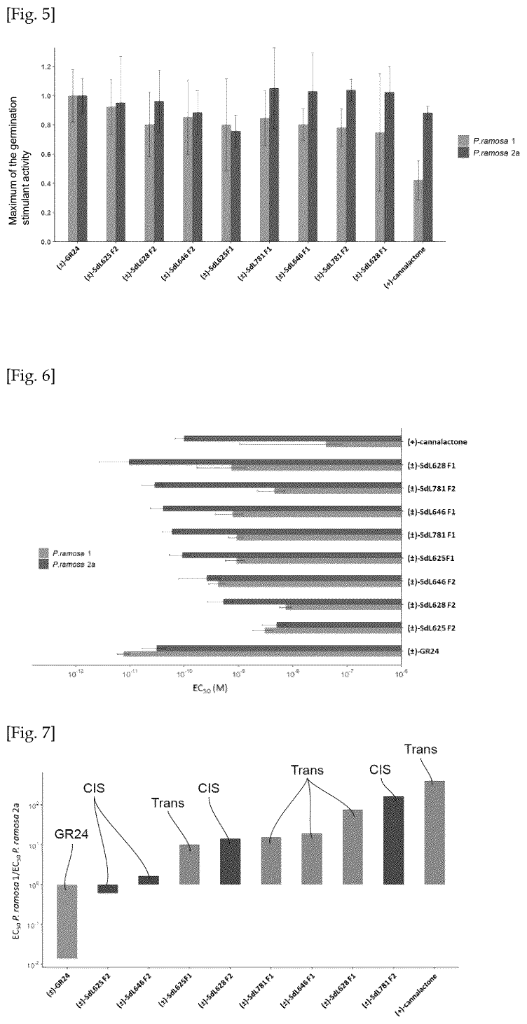

Other characteristics and advantages of the invention may further appear to those skilled in the art upon reading the examples below, given by way of illustration and in a non-limiting manner and shown in the appended figures:

[FIG. 1]-[FIG. 1] shows the diagram of the synthesis of the diastereoisomer (4R*, 6R*)-B21 of Formula (8) carried out in Example 1;

[FIG. 2]-[FIG. 2] shows a global diagram of the analog syntheses of cannalactone performed in Examples 2 to 5, from the diastereoisomer (4R*, 6R*)-B21 of Formula (8) obtained in Example 1;

[FIG. 3]-[FIG. 3] is a schematic representation of the protocol of the germination tests implemented in Example 6.

[FIG. 4]-[FIG. 4] is a dose-response curve of (±)-GR24 and (+)cannalactone on the germination stimulation of P. ramosa 2a and 1 seeds.

[FIG. 5]-[FIG. 5] is a histogram curve showing the maximum activities of stimulation of the germination of the cannalactone analogs according to the invention of Examples 3 to 6 (containing ring A) compared to those of (+)-cannalactone and (±)-GR24 on P. ramosa 1 and 2a seeds. The data are averages±SE (n=6-12 repetitions).

[FIG. 6]-[FIG. 6] is a histogram curve showing the median effective concentrations EC₅₀ (in mol. L⁻¹) of the analogs of cannalactone according to the invention of Examples 3 to 6, compared to those of (+)-cannalactone and (±)-GR24 on the germination stimulation of P. ramosa 1 and 2a seeds. The data are averages±SE (n=6-12 repetitions).

[FIG. 7]-[FIG. 7] is a histogram curve showing the evolution of the ratio of the median effective concentration EC₅₀ of the analogs of cannalactone according to the invention of Examples 3 to 6 (containing ring A) compared to that of (+)cannalactone and (±)-GR24, on the germination stimulation of P. ramosa 1 and 2a seeds.

r_{EC50}=EC₅₀ (P. ramosa 1)/EC₅₀ (P. ramosa 2a) for each analog.

### EXAMPLES

### Solvents and Reagents

Chemical reagents are commercial products marketed in particular by Sigma Aldrich, Alfa Aesar, Acros Organics and TCI. They were used without additional purification.

The analytical-grade anhydrous solvents are commercial products marketed in particular by Sigma Aldrich and Acros Organics.

The tetrahydrofuran (THF) was distilled under argon on sodium in the presence of benzophenone. The deuterated solvents are marketed by Eurisotop.

### Equipment and Methods

The non-aqueous reactions were carried out under an inert atmosphere (argon or nitrogen), using standard techniques for handling compounds sensitive to air and moisture.

All reactions were followed by thin layer chromatography (TLC) on aluminum plates pre-coated with silica gel (marketed by Merck under the trade name 60 F254 with short-wavelength UV detection (i.e., Δ=254 nm), and/or by staining with a solution of KMnO₄ [1% (w/w)] in water or a solution of vanillin [1% (w/w)] in a 1% (v/v) ethanol solution of phosphoric acid.

Most of the separations were performed under silica gel flash chromatography conditions using a filled cartridge (40-63 μm silica gel) at medium pressure (20 psi) with pump and Armen fraction collector or a Buchi Pure C-805 Flash apparatus.

Some separations were carried out on preparative thin layer chromatography (PTLC) (Merck silica gel 60 F254 on glass).

The ¹H NMR spectra were recorded on Bruker spectrometers at 300, 500 or
700 MHz. The ¹³C NMR spectra were recorded on the same instruments at 75,
125 or 175 MHz. Chemical displacements 8 are expressed in parts per million (ppm) with residual solvent signals as internal reference (δ=7.24 for ^{1H} NMR and 77.23 for ^{13C} NMR in CDCl₃). For the ^{1H} NMR, the spectra are described as follows: chemical displacement,
integration, multiplicity (s=singlet, d=doublet, t=triplet, q=quadruplet, quint=quintuplet, sext=sixtuplet, dd=doublet of doublet, dt=doublet of triplet, m=multiplet), coupling constant in Herz (J) and attribution. All NMR attributions are based on COSY, HSQC and HMBC 2D NMR experiments. NOESY experiments were recorded to confirm the configurations of the double bonds.

The IR spectra were recorded on a PerkinElmer Spectrum 100 FT-IR spectrometer, with absorptions given in centimeters⁻¹ (cm⁻¹).

The low resolution mass spectra were determined by electrospray ionization on a Waters Acquity UPLC system, combined with a photodiode detector (PDA), an evaporative light scattering detector (ELSD), and by a mass spectrometer with a tandem quadrupole detector (TQD). The buffers and aqueous mobile phases for UPLC were prepared using water purified with a Milli-Q system.

The high resolution mass spectra were obtained with the Waters Acquity UPLC device (by direct injection or with a BEH C₁₈ 2.1 Å˜50 mm, 1.7 μm column) combined with a PDA and a Waters LCT Premier XE mass instrument [ESI with a Time of Flight (ToF) analyzer].

### Example 1: Synthesis of the Diastereoisomer (4R*, 6R*)-B21 of Formula (8)

### (Access Route Shown in [FIG. 1])

### 4-Bromofuran-2(5H)-one

Oxalyl dibromide (2.6 g, 12.00 mmol, 1.2 equiv.) was added to a solution of furan-2.4 (3H, 5H)-dione (1.0 g, 10.00 mmol) in CH₂Cl₂ (22 mL) and DMF (1 mL) at 0° C. The mixture was stirred for 1 h at 0° C. and gradually heated to room temperature for 2 h. The reaction mixture was diluted with water (50 mL) and extracted with EtOAc (3×20 mL). The combined organic phases were washed with water (2×30 mL), a saturated aqueous solution of NaHCO₃ (2×30 mL) and brine (2×30 mL) and dried over Na₂SO₄. The solvents were removed to obtain the crude product 4-Bromofuran-2(5H)-one (1.61 g, quantitative) in the form of a brown solid. The chemical analyses are in accordance with the literature.

### (4-Bromofuran-2-yl)oxytriisopropylsilane (C4)

Et₃N (626.4 mg, 6.20 mmol, 1.4 equiv.) was added to a solution of 4-bromofuran-2(5H)-one (720.4 mg, 4.40 mmol) in CH₂Cl₂ (6.2 mL) under argon at 0° C. The mixture was stirred for 1 minute, then triisopropylsilyl trifluoromethanesulfonate (TIPSOTf) (1.42 g, 4.60 mmol, 1.05 equiv.) was added dropwise at 0° C. The mixture thus obtained was stirred for 10 minutes at 0° C., then heated to room temperature and stirred for another 1 h 30. The mixture was diluted with heptane (10 mL), washed with a saturated aqueous solution of NaHCO₃ (2×10 mL), water (2×10 mL) and brine (2×10 mL). The organic phase was dried over Na₂SO₄. The solvents were removed to obtain bromofuran C4 (1.4 g, quantitative) in the form of brown oil. The chemical analyses are in accordance with the literature.

### 4-[Hydroxy(8,12,12-trimethylcyclohex-7-en-6-yl)methyl]furan-2(5 H)-one (B20)

To a solution of C4 (89.9 mg, 0.28 mmol) in anhydrous THF (1.8 mL) under
argon at −78° C., a solution of n-BuLi was added dropwise (0.3 mL, 0.30 mmol, 0.98 M, 1.1 equiv.). The mixture thus obtained was stirred at −78° C. for 30 minutes. A mixture of β-cyclocitral (51.6 mg, 0.34 mmol, 1.2 equiv.) in anhydrous THF (2 mL) was then added thereto. The reaction mixture was stirred for 2 h at −78° C. and 12 h at room temperature. The mixture was hydrolyzed with a saturated aqueous solution of NH₄Cl (5 mL) and an aqueous solution of HCl (5 mL, 2 M). The organic phase was separated and the aqueous phase was extracted with EtOAc (3×5 mL). The combined organic phases were washed with water (2×5 mL), a saturated aqueous solution of NaHCO₃ (2×5 mL), water (2×5 mL) and brine (2×5 mL), and then dried over Na₂SO₄. The solvents were removed and the crude product was purified by silica gel chromatography (heptane/EtOAc, 95:5 to 60:40 for 20 min) to obtain the pure product B20 (24.5 mg, 37%) in the form of brown oil:

### B20

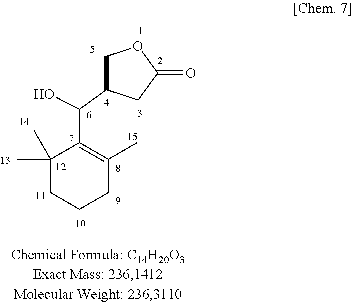

¹H NMR (500 MHz, CDCl₃) δ 5.91 (1H, d, J=1.5 Hz, H-3), 5.10 (1H, s, H-6), 4.88 (1H, d, J=18.0 Hz, H-5a), 4.71 (1H, d, J=18.0 Hz, H-5b), 1.96 (2H, t, J=6.0 Hz, H-9), 1.61 (3H, s, H-15), 1.59-1.55 (2H, m, H-10), 1.50-1.46 (2H, m, H-11), 1.13 (3H, s, H-13 or H-14), 0.98 (3H, s, H-13 or H-14).

¹³C NMR (75 MHz, CDCl₃) δ 174.1 (C-2), 174.0 (C-4), 138.7 (C-7), 136.6 (C-8), 115.0 (C-3), 71.9 (C-5), 67.8 (C-6), 39.5 (C-11), 35.0 (C-12), 33.7 (C-9), 28.9 (C-13 or C-14), 28.5 (C-13 or C-14), 21.4 (C-15), 19.3 (C-10).

IR (film) νₘₐₓ3471, 2932, 1777, 1741, 1637, 1447, 1268, 1111, 1028 cm⁻¹.

HRESIMS m/z 237, 1491 [M+H]⁺ (calc. for C₁₄H₂₁O₃, 237.1491).

### 4-[Hydroxy(8,12,12-trimethylcyclohex-7-en-6-yl)methyl]dihydrofuran-2(3 H)one (B21)

To a solution of B20 (696.4 mg, 2.95 mmol) in methanol (45 mL) at 15° C., NiCl₂ (350.9 mg, 1.48 mmol, 0.5 equiv.) was added then sodium borohydride (358.3 mg, 9.47 mmol, 3.2 equiv.) in portions. The mixture was stirred at 15° C. until the TLC analysis showed complete conversion. The reaction mixture was hydrolyzed with an aqueous solution of HCl (50 mL, 2 M). The aqueous phase was extracted with CH₂Cl₂ (3×20 mL). The combined organic phases were dried over Na₂SO₄ and the solvents were removed. The mixture obtained was then purified by silica gel chromatography (CH₂Cl₂/EtOAc, 100:0 to 90:10 for 30 min) to obtain the pure product (4R*, 6R*)-B21 of Formula 8 (327.4 mg, 47%) in the form of yellow oil and (4R*, 6S*)-B21 of Formula 17 (131.9 mg, 19%) in the form of white solid.

### (4R*, 6R*)-B21

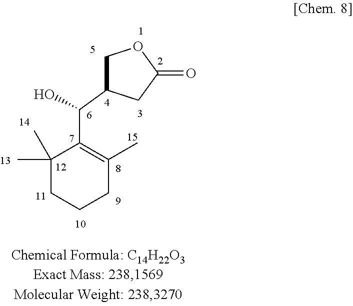

¹H NMR (500 MHz, CDCl₃) δ 4.50 (1H, dd, J=9.5, 7.0 Hz, H-5a), 4.28 (1H, dd, J=9.5, 7.0 Hz, H-5b), 4.21 (1H, d, J=9.5 Hz, H-6), 3.21 (1H, sext, J=9.5 Hz, H-4), 2.41 (1H, dd, J=17.5, 8.5 Hz, H-3a), 2.18 (1H, dd, J=17.5, 8.5 Hz, H-3b), 1.96 (2H, q, J=5.5 Hz, H-9), 1.81 (3H, s, H-15), 1.59-1.52 (2H, m, H-10), 1.48-1.45 (1H, m, H-11a), 1.40-1.35 (1H, m, H-11b), 1.08 (3H, s, H-13 or H-14), 0.98 (3H, s, H-13 or H-14).

¹³C NMR (125 MHz, CDCl₃) δ 177.1 (C-2), 138.4 (C-7), 135.1 (C-8), 72.9 (C-5), 72.6 (C-6), 41.3 (C-4), 40.4 (C-11), 35.0 (C-12), 34.6 (C-9), 32.4 (C-3), 29.2 (C-13 or C-14), 29.1 (C-13 or C-14), 21.3 (C-15), 19.4 (C-10).

IR (film) νₘₐₓ 3464, 2928, 1768, 1551, 1365, 1263, 1178, 1048, 1001, 892 cm⁻¹.

HRESIMS m/z 239.1640 [M+H]⁺ (calc. for C₁₄H₂₃O₃, 239.1647).

### (4R*, 6S*)-B21

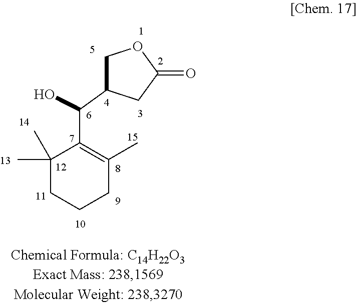

¹H NMR (500 MHz, CDCl₃) δ 4.17 (1H, dd, J=9.0, 7.0 Hz, H-5a), 4.16 (1H, d, J=9.0 Hz, H-6), 3.94 (1H, dd, J=9.0, 7.0 Hz, H-5b), 3.19 (1H, sext, J=9.0 Hz, H-4), 2.72 (1H, dd, J=17.5, 7.5 Hz, H-3a), 2.57 (1H, dd, J=17.5, 7.5 Hz, H-3b), 1.96 (2H, q, J=5.0 Hz, H-9), 1.80 (3H, s, H-15), 1.59-1.51 (2H, m, H-10), 1.47-1.44 (1H, m, H-11a), 1.40-1.34 (1H, m, H-11b), 1.08 (3H, s, H-13 or H-14), 0.97 (3H, s, H-13 or H-14).

¹³C NMR (125 MHz, CDCl₃) δ 177.5 (C-2), 138.2 (C-7), 135.1 (C-8), 72.3 (C-6), 70.5 (C-5), 41.5 (C-4), 40.4 (C-11), 35.1 (C-12), 34.6 (C-9), 33.8 (C-3), 29.2 (C-13 or C-14), 28.8 (C-13 or C-14), 21.4 (C-15), 19.4 (C-10).

IR (film) νₘₐₓ 3481, 2925, 2870, 1774, 1547, 1465, 1373, 1258, 1176, 1092, 1033, 1011, 890, 795 cm⁻¹.

HRESIMS m/z 239.1638 [M+H]⁺ (calc. for C₁₄H₂₃O₃239.1647).

### Example 2: Synthesis of Aromatic Cannalactone Analogs According to the Invention, from the Diastereoisomer (4R*, 6R*)-B21 of Example 1 (of Formula 8)

### (4R*)-[(6R*)-hydroxy(8,12,12-trimethyl-7-oxabicyclo[4.1.0]heptan-6yl)methyl]dihydrofuran-2(3 H)-one((4R*, 6R*)-cis-B22)

This synthesis is shown in [FIG. 2].

To a solution of (4R*, 6R*)-B21 (100.9 mg, 0.420 mmol) in anhydrous toluene (5.1 mL), a solution of VO(acac)₂ (3.9 mg, 0.015 mmol, 0.04 equiv.) in anhydrous toluene (0.2 mL) was added. Tert-Butyl hydroperoxide (TBHP) (0.11 mL, 5.5 M, 0.590 mmol, 1.4 equiv.) The mixture obtained was stirred at room temperature for 1 h. The reaction mixture was hydrolyzed with an aqueous solution of NaOH (5 mL, 5%). The aqueous phase was extracted with heptane and EtOAc (2:1) (3×10 mL). The combined organic phases were washed with brine (2×10 mL), dried over Na₂SO₄ and the solvents were removed to obtain the pure product (4R*, 6R*)-cis-B22 of Formula 9 (116.4 mg, quantitative) in the form of colorless oil used in the following step without purification.

### (4R*, 6R*)-cis-B22

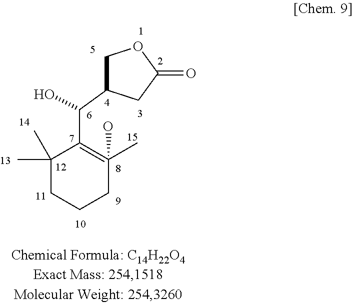

¹H NMR (300 MHz, CDCl₃) δ 4.40 (1H, dd, J=9.5, 8.0 Hz, H-5a), 4.31 (1H, dd, J=9.5, 8.0 Hz, H-5b), 3.96 (1H, d, J=8.0 Hz, H-6), 2.94 (1H, sext, J=8.0 Hz, H-4), 2.59 (1H, dd, J=17.0, 9.0 Hz, H-3a), 2.48 (1H, dd, J=17.0, 9.0 Hz, H-3b), 1.90-1.80 (1H, m, H-9a), 1.78-1.69 (1H, m, H-9b), 1.39 (3H, s, H-15), 1.36-1.32 (2H, m, H-10), 1.25-1.22 (1H, m, H-11a), 1.06 (3H, s, H-13 or H-14), 1.05-1.03 (1H, m, H-11b), 1.02 (3H, s, H-13 or H-14).

¹³C NMR (75 MHz, CDCl₃) δ 176.4 (C-2), 71.1 (C-5), 70.5 (C-6), 70.4 (C-7), 66.3 (C-8), 40.0 (C-4), 37.6 (C-11), 33.9 (C-13), 33.3 (C-3), 31.8 (C-9), 25.6 (C-13 and C-14), 22.2 (C-15), 17.0 (C-10).

IR (film) νₘₐₓ 3464, 2928, 1768, 1551, 1365, 1263, 1178, 1048, 1001, 892 cm⁻¹.

HRESIMS m/z 255.1607 [M+H]⁺ (calc. for C₁₄H₂₃O₄, 255.1596)

### 4-(8,11,12-trimethylbenzyl)dihydrofuran-2(3 H)-one (B38)

To a solution of B22 (62.8 mg, 0.21 mmol) in toluene (15 mL) at 120° C., para-toluene sulfonic acid (APTS) (4.8 mg, 0.02 mmol, 10 mol %) was added and the reaction was stirred for 2 h at this temperature before being cooled to room temperature. The mixture was diluted with water (15 ml), extracted with CH₂Cl₂ (3×10 ml) and dried over Na₂SO₄. The solvents were removed to obtain the crude product B38 (62.1 mg, quantitative) of Formula 10. The crude product was used without purification in the following step.

### B38

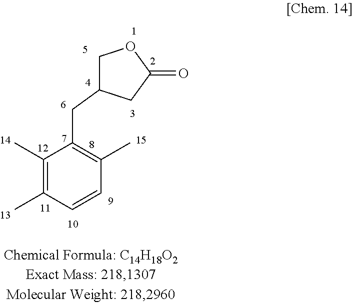 [SMILES: Cc1ccc(C)c(CC2COC(=O)C2)c1C]

¹H NMR (500 MHz, CDCl₃) δ 6.93 (2H, q, J=8.0 Hz, H-9 and H-10), 4.26 (1H, dd, J=9.0, 5.5 Hz, H-5a), 4.02 (1H, dd, J=9.0, 5.5 Hz, H-5b), 2.86-2.84 (2H, m, H-6), 2.78 (1H, sext, J=8.0 Hz, H-4), 2.57 (1H, dd, J=17.0, 8.0 Hz, H-3a), 2.28 (1H, dd, J=17.0, 8.0 Hz, H-3b), 2.27 (3H, s, H-14), 2.23 (3H, s, H-13 or H-15), 2.20 (3H, s, H-13 or H-15).

¹³C NMR (125 MHz, CDCl₃) δ 177.2 (C-2), 135.3 (C-11 or C-8), 135.2 (C-11 or C-8), 135.0 (C-7), 134.1, (C-12), 128.5 (C-9 or C-10), 128.2 (C-9 or C-10), 72.7 (C-5), 36.2 (C-4), 34.7 (C-3), 32.2 (C-6), 21.0 (C-13 or C-15), 20.8 (C-13 or C-15), 16.3 (C-14).

IR (film) νₘₐₓ 2932, 1777, 1734, 1464, 1379, 1169, 1014, 810 cm⁻¹.

HRESIMS m/z 219.1377 [M+H]⁺ (calc. for C₁₄H₁₉O₂, 219.1385).

### (E)-3-(Hydroxymethylene)-4-(8,11,12-trimethylbenzyl)dihydrofuran-2(3 H)one (B40)

To a solution of B38 (62.1 mg, 0.21 mmol) in anhydrous THF (2.1 mL) at 0° C. under argon, ethyl formate (0.16 mL, 2.10 mmol, 10.0 equiv.) and tert-BuOK (235.5 mg, 2.10 mmol, 10.0 equiv.) was added. The mixture was stirred for 30 minutes at 0° C., then allowed to warm to room temperature and stirred for 1 h. The reaction mixture was hydrolyzed with an aqueous solution of HCl (3 mL, 1 M). The mixture was extracted with EtOAc (3×5 mL), washed with brine (2×5 mL), dried over Na₂SO₄ and the solvents were removed. The crude product was purified by silica gel chromatography (heptane/EtOAc, 70:30) to obtain the pure product B40 of Formula 11 (31.4 mg, 61% in 2 steps).

### B40

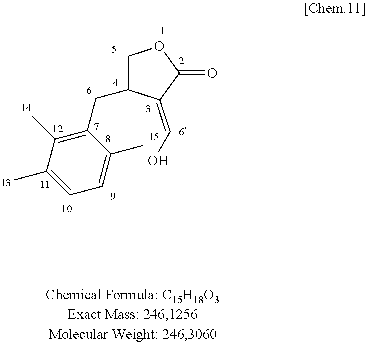 [SMILES: Cc1ccc(C)c(CC2COC(=O)/C2=C/O)c1C]

IR (film) νmax 3673, 2969, 2922, 1778, 1745, 1462, 1385, 1262, 1169, 1051, 799 cm⁻¹.

HRESIMS m/z 245.1176 [M−H]⁺ (calc. for C15H17O3, 245.1178).

### (±)-SdL625

To a solution of B40 (30.0 mg, 0.12 mmol) in anhydrous acetone (1.2 mL) under argon, anhydrous K₂CO₃ (34.5 mg, 0.24 mmol, 2.0 equiv.) was added. To this mixture was added 5-bromo-3-methylfuran-2(5H)-one D4 (32.3 mg, 0.18 mmol, 1.5 equiv.) in anhydrous acetone (1.2 ml). The reaction was stirred for 2 h at room temperature. The solvents were removed and the mixture was dissolved in EtOAc (5 mL) and filtered to remove the salts. The solvents were removed and the crude product was purified by PTLC (heptane/EtOAc, 50:50) to obtain the pure product (±)-SdL625 F1 of Formula 18 (8.3 mg, 17%) and (±)-SdL625 F2 of Formula 19 (7.1 mg, 20%) in the form of colorless oils. (±)-SdL625 F1 and (±)-SdL625 F2 are aromatic-type cannalactone analogs corresponding to general Formula 2.

### (±)-SdL625 F1

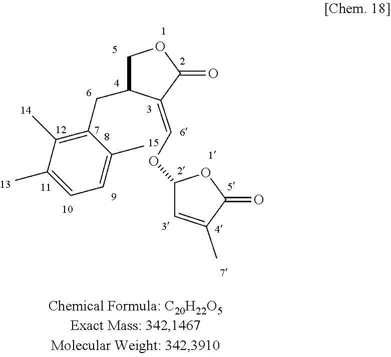

¹H NMR (700 MHz, CDCl3) δ 7.41 (1H, d, J=1.5 Hz, H-6′), 6.94 (1H, d, J=8.0 Hz, H-9 or H-10), 6.90 (1H, d, J=8.0 Hz, H-9 or H-10), 6.57 (1H, t, J=1.5 Hz, H-3′), 5.81 (1H, t, J=1.5 Hz, H-2′), 4.16 (1H, dd, J=9.0, 7.0 Hz, H-5a), 4.07 (1H, dd, J=9.0, 1.5 Hz, H-5b), 3.50 (1H, q, J=7.5 Hz, H-4), 3.01 (1H, dd, J=14.0, 8.5 Hz, H-6a), 2.88 (1H, dd, J=14.0, 8.5 Hz, H-6a), 2.26 (3H, s, H-13 or H-15), 2.23 (3H, s, H-13 or H-15), 2.20 (3H, s, H-14), 1.97 (3H, s, H-7′).

¹³C NMR (175 MHz, CDCl3) δ 171.9 (C-2), 170.4 (C-5′), 150.9 (C-6′), 141.1 (C-3′), 135.6 (C-4′), 135.6 (C-12), 135.3 (C-7), 134.8 (C-8 or C-11), 134.7 (C-8 or C-11), 128.3 (C-9 or C-10), 127.9 (C-9 or C-10), 112.2 (C-3), 100.3 (C-2′), 71.0 (C-5), 37.1 (C-4), 33.0 (C-6), 21.0 (C-13 or C-15), 21.0 (C-13 or C-15), 16.4 (C-14), 10.9 (C-7′).

IR (film) νmax 2969, 2924, 2860, 1785, 1754, 1681, 1465, 1340, 1257, 1174, 1084, 1021, 953, 868, 794 cm⁻¹.

HRESIMS m/z 343.1538 [M+H]⁺ (calc. for C20H23O5, 343.1545).

### (±)-SdL625 F2

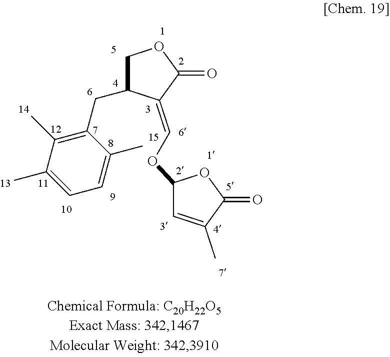

¹H NMR (700 MHz, CDCl3) δ 7.38 (1H, s, H-6′), 6.88 (1H, d, J=8.0 Hz, H-9 or H-10), 6.84 (1H, d, J=8.0 Hz, H-9 or H-10), 6.68 (1H, s, H-3′), 5.93 (1H, s, H-2′), 4.15 (1H, dd, J=9.5, 7.0 Hz, H-5a), 4.07 (1H, dd, J=9.5, 2.0 Hz, H-5b), 3.50 (1H, q, J=8.0 Hz, H-4), 3.02 (1H, dd, J=14.0, 7.5 Hz, H-6a), 2.86 (1H, dd, J=14.0, 9.5 Hz, H-6a), 2.24 (3H, s, H-13 or H-15), 2.20 (3H, s, H-13 or H-15), 2.19 (3H, s, H-14), 1.99 (3H, s, H-7′).

¹³C NMR (175 MHz, CDCl3) δ 171.9 (C-2), 170.2 (C-5′), 150.3 (C-6′), 140.7 (C-3′), 136.1 (C-4′), 135.4 (C-12), 135.1 (C-7), 134.8 (C-8 or C-11), 134.5 (C-8 or C-11), 128.3 (C-9 or C-10), 127.9 (C-9 or C-10), 112.1 (C-3), 100.0 (C-2′), 71.0 (C-5), 37.1 (C-4), 32.9 (C-6), 21.0 (C-13 or C-15), 21.0 (C-13 or C-15), 16.3 (C-14), 11.0 (C-7′).

IR (film) νmax 2966, 2922, 2848, 1781, 1756, 1682, 1347, 1260, 1184, 1090, 1024, 950, 797 cm⁻¹.

HRESIMS m/z 343.1540 [M+H]⁺ (calc. for C20H23O5, 343.1545).

### Example 3: Synthesis of Diene-Type Cannalactone Analogs According to the Invention, from the Diastereoisomer (4R*, 6R*)-B21 of Example 1

This synthesis is shown in [FIG. 2].

### (E)-4-[(8,12,12-Trimethylcyclohex-8-en-6-ylidene)methyl]dihydrofuran-2(3 H)-one ((E)-B25)

To a solution of B21 (200.0 mg, 0.84 mmol) in pyridine (6.8 mL) was added DMAP (4-dimethylaminopyridine 5.1 mg, 0.04 mol, 5 mol %) and MsCl (0.3 mL, 3.40 mmol, 4.0 equiv.). The mixture was stirred for one night at room temperature. The reaction mixture was co-evaporated with toluene. The mixture was diluted with CH₂Cl₂ (10 mL), washed with water (2×5 mL) and brine (2×5 mL) and dried with Na₂SO₄. The solvents were removed and the crude product was purified by silica gel chromatography (heptane/EtOAc, 80:20) to obtain the pure product (E)-B25 of Formula 13 (142.2 mg, 77%).

### (E)-B25

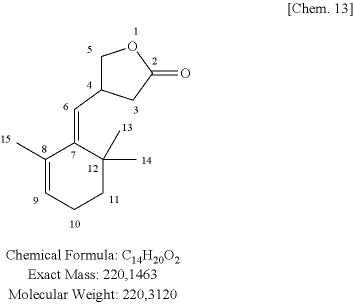 [SMILES: CC1=CCCC(C)(C)/C1=C\C1COC(=O)C1]

¹H NMR (500 MHz, CDCl3) δ 5.73 (1H, t, J=4.5 Hz, H-9), 5.18 (1H, d, J=10.0 Hz, H-6), 4.44 (1H, t, J=8.0 Hz, H-5a), 3.93 (1H, t, J=8.0 Hz, H-5b), 3.69 (1H, m, H-4), 2.69 (1H, dd, J=17.5, 8.0 Hz, H-3a), 2.30 (1H, dd, J=17.0, 9.5 Hz, H-3b), 2.05 (1H, m, 2 H-11), 1.78 (3H, s, H-15), 1.46 (2H, t, J=5.6 Hz, H-10), 1.20 (6H, s, H-13 and H-14).

¹³C NMR (125 MHz, CDCl3) δ 176.9 (C-2), 147.4 (C-7), 132.8 (C-8), 128.5 (C-9), 122.8 (C-6), 73.8 (C-5), 40.3 (C-11), 36.7 (C-4), 36.5 (C-3), 35.0 (C-12), 29.5 (C-13 or C-14), 29.1 (C-13 or C-14), 22.9 (C-10), 22.0 (C-15).

IR (film) νmax 2932, 2857, 1779, 1545, 1469, 1380, 1265, 1178, 1042, 1001, 882, 739 cm⁻¹.

HRESIMS m/z 221.1542 [M+H]⁺ (calc. for C14H21O2, 221.1542).

### (E)-3-(Hydroxymethylene)-4-[(E)-(8,12,12-trimethylcyclohex-8-en-6-ylidene)methyl]dihydrofuran-2(3 H)-one (B42)

To a solution of (E)-B25 (19.0 mg, 0.09 mmol) in anhydrous THF (0.9 mL) at 0° C. under argon, is added ethyl formate (70 μL, 0.90 mmol, 10.0 equiv.) and tert-BuOK (101.0 mg, 0.90 mmol, 10.0 equiv.). The mixture was stirred for 30 minutes at 0° C., then allowed to heat to room temperature and stirred for 1 h. The reaction mixture was hydrolyzed with an aqueous solution of HCl (1 mL, 1 M). The mixture was extracted with EtOAc (5 mL), washed with brine (2×5 mL) and dried on Na₂SO₄. The solvents were removed and the crude product was purified by silica gel chromatography (heptane/EtOAc, 70:30) to obtain the pure product B42 of Formula 14 (14.8 mg, 66%) in the form of colorless oil.

### B42

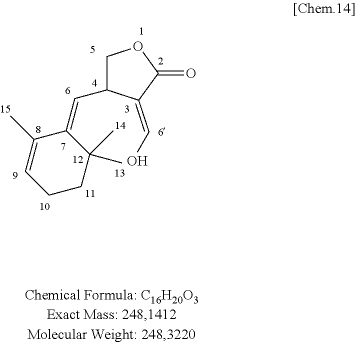 [SMILES: CC1=CCCC(C)(C)/C1=C\C1COC(=O)/C1=C/O]

IR (film) νmax 3664, 2975, 2919, 1734, 1396, 1056 cm⁻¹.

HRESIMS m/z 247.1332 [M+H]⁺ (calc. for C15H19O3, 247.1334).

### (±)-SdL646

To a solution of B42 (27.7 mg, 0.11 mmol) in anhydrous acetone (1.1 mL) under argon, anhydrous K₂CO₃ (32.3 mg, 0.22 mmol, 2.0 equiv.) was added. To this mixture was added 5-bromo-3-methylfuran-2(5H)-one D4 (30.1 mg, 0.17 mmol, 1.5 equiv.) in anhydrous acetone (1.1 ml). The reaction was stirred for 2 h at room temperature. The solvents were removed and the crude product was dissolved in EtOAc (5 mL) and filtered to remove salts. The solvents were removed and the crude product was purified by PTLC (heptane/EtOAc, 50:50) to obtain the pure product (±)-SdL646 F1 of Formula 20 (10.9 mg, 19%) and (±)-SdL646 F2 of Formula 21 (7.3 mg, 29%) in the form of colorless oils. (±)-SdL646 F1 and (±)SdL646 F2 are diene-type cannalactone analogs corresponding to general Formula 3.

### (±)-SdL646 F1

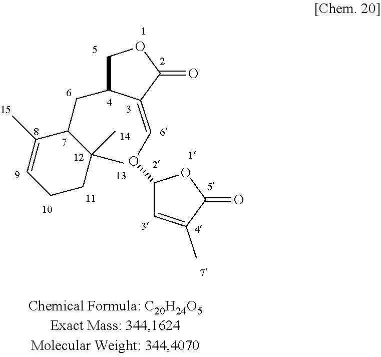

¹H NMR (700 MHz, CDCl₃) δ 7.48 (1H, d, J=2.0 Hz, H-6′), 6.80 (1H, t, J=1.5 Hz, H-3′), 6.06 (1H, s, H-2′), 5.70 (1H, t, J=4.5 Hz, H-9), 5.23 (1H, d, J=10.0 Hz, H-6), 4.51 (1H, q, J=8.5 Hz, H-5a), 4.49-4.47 (1H, m, H-4), 3.98 (1H, dd, J=8.5, 4.5 Hz, H-5b), 2.11-2.05 (2H, m, H-10), 1.97 (3H, s, H-7′), 1.75 (3H, s, H-15), 1.54-1.50 (1H, m, H-11a), 1.39-1.36 (1H, m, H-11b), 1.20 (3H, s, H-13 or H-14), 1.13 (3H, s, H-13 or H-14).

¹³C NMR (175 MHz, CDCl₃) δ 171.8 (C-2), 170.4 (C-5′), 151.2 (C-6′), 145.3 (C-7), 141.0 (C-3′), 135.9 (C-4′), 133.0 (C-8), 127.8 (C-9), 123.4 (C-6), 112.5 (C-3), 100.5 (C-2′), 72.5 (C-5), 40.3 (C-11), 37.4 (C-4), 34.7 (C-12), 30.8 (C-13 or C-14), 27.2 (C-13 or C-14), 22.9 (C-10), 22.1 (C-15), 10.9 (C-7′).

IR (film) νₘₐₓ 2969, 2925, 2848, 1782, 1757, 1679, 1471, 1344, 1184, 1088, 1029, 1007, 953 cm⁻¹.

HRESIMS m/z 345.1697 [M+H]⁺ (calc. for C₂₀H₂₅O₅, 345.1702).

### (±)-SdL646 F2

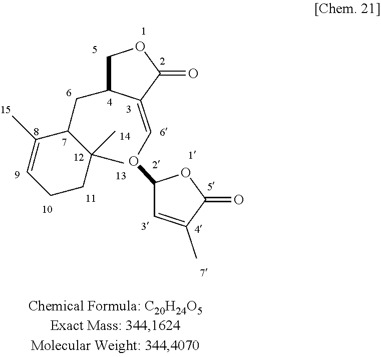

¹H NMR (700 MHz, CDCl₃) δ 7.45 (1H, d, J=2.5 Hz, H-6′), 6.81 (1H, t, J=1.5 Hz, H-3′), 6.07 (1H, t, J=1.5 Hz, H-2′), 5.67 (1H, t, J=4.0 Hz, H-9), 5.22 (1H, d, J=10.0 Hz, H-6), 4.51 (1H, q, J=8.5 Hz, H-5a), 4.49-4.46 (1H, m, H-4), 3.97 (1H, dd, J=8.5, 5.5 Hz, H-5b), 2.09-2.04 (2H, m, H-10), 1.96 (3H, s, H-7′), 1.70 (3H, s, H-15), 1.54-1.50 (1H, m, H-11a), 1.38-1.35 (1H, m, H-11b), 1.21 (3H, s, H-13 or H-14), 1.15 (3H, s, H-13 or H-14).

¹³C NMR (175 MHz, CDCl3) δ 171.8 (C-2), 170.3 (C-5′), 151.0 (C-6′), 145.4 (C-7), 141.0 (C-3′), 136.0 (C-4′), 133.1 (C-8), 127.5 (C-9), 123.2 (C-6), 112.7 (C-3), 100.3 (C-2′), 72.3 (C-5), 40.3 (C-11), 37.6 (C-4), 34.7 (C-12), 30.9 (C-13 or C-14), 27.1 (C-13 or C-14), 22.9 (C-10), 21.9 (C-15), 10.9 (C-7′).

IR (film) νmax 2963, 2922, 2851, 1782, 1756, 1679, 1453, 1341, 1260, 1184, 1084, 1025, 1009, 953 cm⁻¹.

HRESIMS m/z 345.1703 [M+H]⁺ (calc. for C20H25O5, 345.1702).

### Example 4: Synthesis of Silylated-Type Cannalactone Analogs According to the Invention, from the Diastereoisomer (4R*, 6R*)-B21 of Example 1

This synthesis is shown in [FIG. 2].

### (4R*)-[(6R*)-(8,12,12-trimethylcyclohex-7-en-6-yl)((trimethyl silyl)oxy)methyl]dihydrofuran-2(3 H)-one (4R*, 6R*)-B45a

A solution of (4R*, 6R*)-B21 (111.1 mg, 0.47 mmol) in TMS-imidazole (2.1 mL, 14.00 mmol, 30.0 equiv.) was stirred for 1 h at 50° C. The reaction mixture was cooled to room temperature and stirred for 1 h. The mixture was dissolved with petroleum ether (5 mL), washed with brine (2×5 mL), dried over Na₂SO₄ and the solvents were removed to obtain the crude product (4R*, 6R*)-B45a of Formula 15 with R⁴ denoting a methyl group (135.5 mg, quantitative) in the form of a colorless oil. The crude product was used without purification in the following step.

### (4R*, 6R*)-B45a

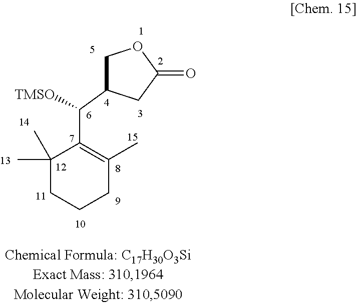

¹H NMR (500 MHz, DMSO-d₆) δ 4.63-4.54 (1H, m, H-6), 4.33 (1H, t, J=8.0 Hz, H-5a), 4.17 (1H, t, J=8.0 Hz, H-5b), 3.00-2.95 (1H, m, H-4), 2.45 (1H, dd, J=17.0, 8.5, Hz, H-3a), 2.20-2.11 (1H, m, H-3b), 1.99 (2H, t, J=6.5 Hz, H-9), 1.72 (3H, s, H-15), 1.61 (2H, quint, J=6.5 Hz, H-10), 1.40-1.38 (2H, m, H-11), 1.13 (3H, s, H-13 or H-14), 1.09 (3H, s, H-13 or H-14), 0.10 (9H, s, H-TMS).

¹³C NMR (125 MHz, DMSO-d₆) δ 175.8 (C-2), 137.6 (C-7), 130.7 (C-8), 71.5 (C-6), 70.2 (C-5), 41.6 (C-4), 41.0 (C-11), 33.2 (C-9), 31.3 (C-3), 28.8 (C-12), 28.4 (C-13 or C-14), 28.3 (C-13 or C-14), 20.2 (C-15), 18.0 (C-10), 0.32 (C-TMS).

IR (film) νₘₐₓ 2925, 2850, 1782, 1465, 1253, 1173, 1067, 883, 839, 747 cm⁻¹.

HRESIMS m/z 311.2036 [M+H]⁺ (calc. for C₁₇H₃₁O₃Si, 311.2042).

### (4R*)-3-(E)-(Hydroxymethylene)-4-[(6R*)-(8,12,12-trimethylcyclohex-7-ene yl)((trimethylsilyl)oxy)methyl]dihydrofuran-2(3 H)-one ((4R*, 6R*)-B46a)

To a solution of (4R*, 6R*)-B45a (13.0 mg, 0.04 mmol) in anhydrous THF (0.4 mL) at −40° C. under argon, ethyl formate (32 μL, 0.40 mmol, 10.0 equiv.) and the tert-BuOK (33.3 mg, 0.28 mmol, 7.0 equiv.) was added. The mixture was stirred for 1 h at −40° C., then heated to −10° C. and stirred for an additional 1 h. The reaction medium was diluted with EtOAc (5 mL), washed with water (2×5 mL) and an aqueous saturated NH₄Cl solution (2×5 mL). The organic phase was dried over Na₂SO₄ and concentrated under reduced pressure to obtain the desired crude product (4R*, 6R*)-B46a of Formula 16 with R⁴ denoting a methyl group (10.6 mg). The crude product was used without any purification in the following step.

### (4R*, 6R*)-B46a

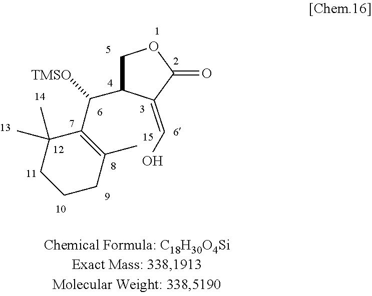

IR (film) νₘₐₓ 3464, 2925, 1763, 1462, 1379, 1253, 1219, 1178, 1067, 977, 839 cm⁻¹.

HRESIMS m/z 339.2001 [M+H]⁺ (calc. for C₁₈H₃₁O₄Si, 339.1992).

### (±)-SdL781

To a solution of (4R*, 6R*)-B46a (28.1 mg, 0.08 mmol) in anhydrous THF (0.8 mL) at −78° C. under argon, tert-BuOK (14.1 mg, 0.12 mmol, 1.5 equiv.) was added. To this mixture was added 5-bromo-3-methylfuran-2(5H)-one D4 (21.2 mg, 0.12 mmol, 1.5 equiv.) in anhydrous THF (0.8 mL). The reaction medium was heated to room temperature and stirred for one night. The reaction mixture was dissolved in EtOAc (5 mL), washed with water (2×5 mL) and brine (2×5 mL) and dried over Na₂SO₄. The solvents were removed and the crude product was purified by PTLC (petroleum ether/EtOAc, 60:40) to obtain the products (±)-SdL781 F1 of Formula 22 (14.7 mg, 32% in 3 steps) and (±)-SdL781 F2 of Formula 23 (14.0 mg, 31% in 3 steps). (±)-SdL781 F1 and (±)-SdL781 F2 are silyl-type cannalactone analogs corresponding to general Formula 4 with R⁴ denoting a methyl group.

### (±)-SdL781 F1

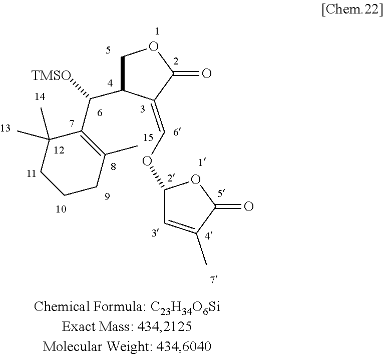

¹H NMR (500 MHz, CDCl₃) δ 7.45 (1H, s, H-6′), 6.89 (1H, s, H-3′), 6.09 (1H, s, H-2′), 4.59-4.51 (2H, m, H-6 and H-5a), 4.13 (1H, t, J=8.0 Hz, H-5b), 3.60-3.54 (1H, m, H-4), 2.00 (3H, s, H-7′), 1.88-1.81 (2H, m, H-9), 1.53-1.46 (2H, m, H-10), 1.33-1.27 (2H, m, H-11), 1.23 (3H, s, H-15), 1.08 (3H, s, H-13 or H-14), 0.99 (3H, s, H-13 or H-14), 0.05 (9H, s, H-TMS).

¹³C NMR (125 MHz, CDCl₃) δ 172.4 (C-2), 170.2 (C-5′), 148.6 (C-6′), 142.0 (C-7), 140.9 (C-3′), 136.2 (C-4′), 136.2 (C-12), 100.7 (C-2′), 96.9 (C-3), 72.2 (C-6), 69.2 (C-5), 45.1 (C-4), 40.9 (C-9), 34.7 (C-11), 29.9 (C-15), 29.9 (C-8), 29.3 (C-13 and C-14), 19.1 (C-10), 11.9 (C-7′), 0.5 (C-TMS).

IR (film) νₘₐₓ 2954, 2920, 2853, 1784, 1755, 1686, 1462, 1342, 1254, 1191, 1082, 1026, 955, 887, 843, 752 cm⁻¹.

HRESIMS m/z 435.2189 [M+H]⁺ (calc. for C₂₃H₃₅O₆Si, 435.2203).

### (±)-SdL781 F2

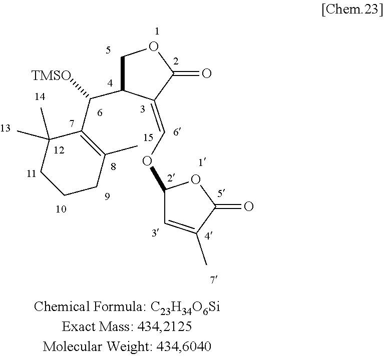

¹H NMR (500 MHz, CDCl₃) δ 7.39 (1H, s, H-6′), 6.87 (1H, s, H-3′), 6.11 (1H, s, H-2′), 4.61 (1H, d, J=5.5 Hz, H-6), 4.56 (1H, d, J=8.5 Hz, H-5a), 4.14 (1H, t, J=8.5 Hz, H-5b), 3.58-3.53 (1H, m, H-4), 2.01 (3H, s, H-7′), 1.92 (2H, t, J=7.0 Hz, H-9), 1.61-1.52 (2H, m, H-10), 1.42-1.34 (2H, m, H-11), 1.23 (9H, s, H-13, H-14 and H-15), 0.05 (9H, s, H-TMS).

¹³C NMR (175 MHz, CDCl₃) δ 172.3 (C-2), 170.3 (C-5′), 149.6 (C-6′), 140.9 (C-7), 140.8 (C-3′), 136.4 (C-4′), 135.9 (C-12), 100.4 (C-2′), 96.2 (C-3), 72.0 (C-6), 69.0 (C-5), 45.1 (C-4), 41.0 (C-9), 34.7 (C-11), 29.9 (C-13 and C-14), 29.6 (C-8), 29.4 (C-15), 19.3 (C-10), 11.0 (C-7′), 0.4 (C-TMS).

IR (film) νₘₐₓ 2959, 2923, 2853, 1788, 1755, 1683, 1463, 1342, 1252, 1209, 1180, 1085, 1024, 958, 887, 842, 752 cm⁻¹.

HRESIMS m/z 435.2188 [M+H]⁺ (calc. for C₂₃H₃₅O₆Si, 435.2203).

### Example 5: Synthesis of an Alcohol-Type Cannalactone Analog According to the Invention, from the Diastereoisomer (4R*, 6R*)-B21 of Example 1

This synthesis is shown in [FIG. 2].

### (4R*)-[(6R*)-{(Triethylsilyl)oxy}(8,12,12-trimethylcyclohex-7-en-6yl)methyl]dihydrofuran-2(3 H)-one ((4R*, 6R*)-B45b)

To a solution of (4R*, 6R*)-B21 (12.4 mg, 0.05 mmol) in pyridine (0.4 mL) was added 4-dimethylamino pyridine (DMAP) (1.9 mg, 2 μmol, 0.3 equiv.) and triethylsilyl chloride (TESCl) (50 μL, 0.30 mmol, 6.0 equiv.). The mixture was stirred for 24 h. The reaction mixture was dissolved with CH₂Cl₂ (5 mL), washed with a saturated aqueous solution of NaHCO₃ (2×5 mL) and dried over Na₂SO₄. The solvents were removed and the crude product was purified by silica column chromatography (petroleum ether/EtOAc, 100:0 to 80:20 for 10 min) to obtain the pure product (4R*, 6R*)-B45b of Formula 15 with R⁴ denoting an ethyl group (12.3 mg, 70%) in the form of two conformers as a colorless oil.

### (4R*, 6R*)-B45b

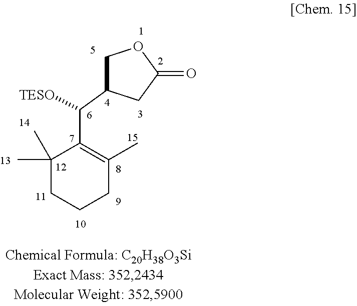

### Conformer 1:

¹H NMR (500 MHz, DMSO-d6) δ 4.71 (1H, d, J=11.0 Hz, H-6), 4.40 (1H, t, J=8.0 Hz, H-5a), 4.22 (1H, q, J=4.5 Hz, H-5b), 3.06-3.00 (1H, m, H-4), 2.06 (2H, dd, J=16.5, 6.5 Hz, H-3), 2.02-1.97 (2H, m, H-9), 1.68-1.64 (2H, m, H-10), 1.63 (3H, s, H-15), 1.43-1.37 (2H, m, H-11), 1.17 (3H, s, H-13 or H-14), 1.10 (3H, s, H-13 or H-14), 0.92 (9H, t, J=7.0 Hz, H-CH3-TES), 0.58 (6H, q, J=7.0 Hz, H-CH2-TES).

¹³C NMR (125 MHz, DMSO-d6) δ 177.5 (C-2), 136.4 (C-7), 132.3 (C-8), 74.4 (C-6), 72.6 (C-5), 42.4 (C-4, C-11), 34.4 (C-12), 33.4 (C-9), 32.1 (C-3), 30.7 (C-13 or C-14), 30.6 (C-13 or C-14), 21.0 (C-15), 18.9 (C-10), 7.16 (C-CH3-TES), 5.3 (C-CH2-TES).

### Conformer 2:

¹H NMR (500 MHz, DMSO-d₆) δ 4.31 (2H, d, J=5.5.0 Hz, H-5), 4.27 (1H, d, J=8.5 Hz, H-6), 2.99-2.94 (1H, m, H-4), 2.29 (2H, d, J=9.5 Hz, H-3), 1.96-1.85 (2H, m, H-9), 1.78 (3H, s, H-15), 1.59-1.51 (2H, m, H-10), 1.51-1.37 (1H, m, H-11a), 1.34-1.30 (1H, m, H-11b), 1.08 (3H, s, H-13 or H-14), 0.90 (3H, s, H-13 or H-14), 0.92 (9H, t, J=7.0 Hz, H-CH₃-TES), 0.58 (6H, q, J=7.0 Hz, H-CH₂-TES).

¹³C NMR (125 MHz, DMSO-d₆) δ 177.2 (C-2), 137.0 (C-7), 133.6 (C-8), 71.5 (C-6), 71.2 (C-5), 44.1 (C-4), 40.6 (C-11), 34.7 (C-12), 34.6 (C-9), 33.1 (C-3), 30.2 (C-13 or C-14), 29.2 (C-13 or C-14), 21.9 (C-15), 19.4 (C-10), 7.16 (C-CH₃-TES), 5.5 (C-CH₂TES).

IR (film) νₘₐₓ 2963, 2928, 2881, 1782, 1666, 1460, 1412, 1371, 1241, 1175, 1069, 1006, 819, 741 cm⁻¹

HRESIMS m/z 353.2511 [M+H]⁺ (calc. for C₂₀H₃₇O₃Si, 353.2512).

### (4R*, 6R*)-B48

To a solution of (4R*, 6R*)-B45b (21.5 mg, 0.06 mmol) in anhydrous THF (0.5 mL) at −40° C. under argon, ethyl formate (50 μL, 0.60 mmol, 10.0 equiv.) and tert-BuOK (47.1 mg, 0.42 mmol, 7.0 equiv.) were added. The mixture was stirred for 1 h at 0° C. and then cooled to −78° C. 5-bromo-3-methylfuran-2(5H)-one D4 [16 (15.9 mg, 0.09 mmol, 1.5 equiv.) was added to this mixture in anhydrous THF (0.5 mL). The reaction medium was heated to room temperature and stirred for one night. The mixture was dissolved in EtOAc (5 mL), washed with water (2×5 mL) and brine (2×5 mL) and dried over Na₂SO₄. The solvents were removed and the crude product was purified by silica gel column chromatography (heptane/EtOAc, 100:0 to 70:30) to obtain the product (4R*, 6R*)-B48 F1 of Formula 24 (6.1 mg, 21%) and (4R*, 6R*)-B48 F2 of Formula 25 (7.0 mg, 25%).

### (4R*, 6R*)-B48 F1

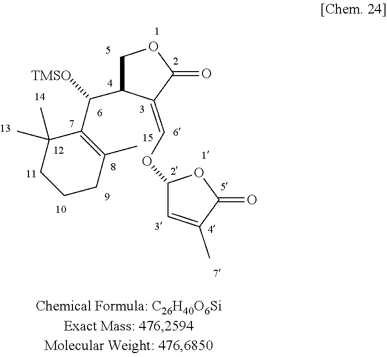

¹H NMR (500 MHz, CDCl₃) δ 7.40 (1H, s, H-6′), 6.86 (1H, t, J=1.5 Hz, H-3′), 6.11 (1H, s, H-2′), 4.64-4.59 (1H, m, H-5a), 4.58-4.54 (1H, m, H-6), 4.16 (1H, t, J=8.5 Hz, H-5b), 3.57-3.53 (1H, m, H-4), 2.01 (3H, t, J=1.5 Hz, H-7′), 1.91 (2H, t, J=6.5 Hz, H-9), 1.59-1.54 (2H, m, H-10), 1.39-1.35 (2H, m, H-11), 1.19 (3H, s, H-15), 1.06 (3H, s, H-13 or H-14), 0.92 (3H, s, H-13 or H-14), 0.90 (9H, t, J=7.5 Hz, H-CH₃-TES), 0.55 (6H, q, J=7.5 Hz, H-CH₂-TES).

¹³C NMR (125 MHz, CDCl₃) δ 172.3 (C-2), 170.3 (C-5′), 149.9 (C-6′), 140.8 (C-3′), 136.6 (C-12), 136.4 (C-4′), 133.4 (C-7), 102.0 (C-3), 100.5 (C-2′), 71.9 (C-6), 69.0 (C-5), 45.3 (C-4), 40.8 (C-9), 34.9 (C-11), 32.1 (C-8), 30.1 (C-13 or C-14), 29.9 (C-15), 29.5 (C-13 or C-14), 19.3 (C-10), 11.0 (C-7′), 7.1 (C-CH₃-TES), 5.3 (C-CH₂TES).

IR (film) νₘₐₓ 2963, 2922, 1787, 1757, 1681, 1466, 1343, 1259, 1184, 1084, 1018, 951, 862, 800, 743 cm⁻¹.

HRESIMS m/z 477.2659 [M+H]⁺ (calc. for C₂₆H₄₁O₆Si, 477.2672).

### (4R*, 6R*)-B48 F2

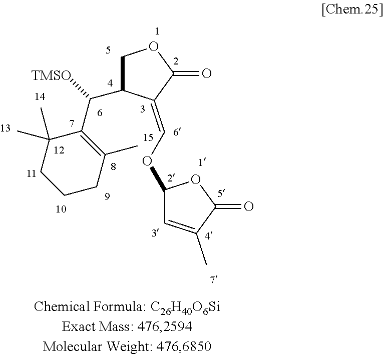

¹H NMR (500 MHz, CDCl₃) δ 7.46 (1H, s, H-6′), 6.88 (1H, t, J=1.5 Hz, H-3′), 6.09 (1H, s, H-2′), 4.62-4.58 (1H, m, H-5a), 4.88 (1H, s, H-6), 4.17-4.13 (1H, m, H-5b), 3.59-3.54 (1H, m, H-4), 2.01 (3H, s, H-7′), 1.88-1.83 (2H, m, H-9), 1.52-1.45 (2H, m, H-10), 1.32-1.323 (2H, m, H-11), 1.23 (3H, s, H-15), 1.02 (3H, s, H-13 or H-14), 0.93 (3H, s, H-13 or H-14), 0.90 (9H, t, J=7.0 Hz, H-CH₃-TES), 0.54 (6H, q, J=7.5 Hz, H-CH₂-TES).

¹³C NMR (125 MHz, CDCl₃) δ 172.3 (C-2), 170.2 (C-5′), 150.5 (C-6′), 140.8 (C-3′), 136.6 (C-12), 136.2 (C-4′), 133.5 (C-7), 100.6 (C-2′), 100.1 (C-3), 71.8 (C-6), 69.3 (C-5), 45.3 (C-4), 40.7 (C-9), 34.8 (C-11), 32.1 (C-8), 29.9 (C-13 or C-14), 29.9 (C-15), 29.5 (C-13 or C-14), 19.2 (C-10), 10.9 (C-7′), 7.1 (C-CH₃-TES), 5.3 (C-CH₂TES).

IR (film) νₘₐₓ 2960, 2922, 1788, 1753, 1679, 1460, 1259, 1184, 1091, 1015, 868, 797 cm⁻¹.

HRESIMS m/z 477.2564 [M+H]⁺ (calc. for C₂₆H₄₁O₆Si, 477.2672).

### (±)-SdL628 F1

### Method 1

To a solution of (±)-SdL781 F1 (14.3 mg, 0.032 mmol) in CH₃CN (0.2 mL) and water (2 drops) was added a solution of Sc(OTf)₃ (0.1 mg, 16 μmol, 0.5 mol %) in CH₃CN (0.2 mL). The resulting mixture was stirred for 1 h 30 at room temperature and hydrolyzed with an aqueous phosphate buffer (2 mL, pH 7). The organic phase was extracted with CH₂Cl₂ (3×2 mL), and the combined extracts were washed with brine (2×3 mL), then dried over Na₂SO₄. The solvents were removed and the crude product was purified by PTLC (petroleum ether/EtOAc, 60:40) to obtain the product (±)-SdL628 F1 of Formula 26 (2.4 mg, 22%) in the form of colorless oil.

### Method 2

To a solution of (4R*, 6R*)-B48 F1 (6.1 mg, 0.01 mmol) in anhydrous THF (1 mL) under argon was added a solution of 3HF·NET₃ (20 μL, 0.13 mmol, 10.0 equiv.). The mixture was stirred for one night at 50° C. The organic phase was cooled with a saturated aqueous solution of NaHCO₃ (1 mL) and extracted with EtOAc (3×2 mL). The organic phase was dried over Na₂SO₄ and the solvents were removed. The crude product was purified by PTLC (heptane/EtOAc, 60:40) to obtain the product (±)-SdL628 F1 of Formula 26 (3.9 mg, 83%) in the form of colorless oil.

### (±)-SdL628 F1

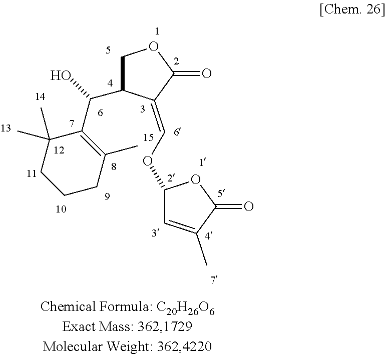

¹H NMR (500 MHz, CDCl3) δ 7.35 (1H, s, H-6′), 6.82 (1H, s, H-3′), 6.06 (1H, s, H-2′), 4.60 (1H, d, J=10.0 Hz, H-6), 4.23 (1H, dd, J=9.0, 6.0 Hz, H-5a), 4.17 (1H, d, J=10.0 Hz, H-5b), 3.86-3.82 (1H, m, H-4), 1.83 (3H, S, H-7′), 1.82-178 (2H, m, H-9), 1.43-1.40 (2H, m, H-10), 1.39-1.34 (2H, m, H-11), 1.23 (3H, s, H-15), 1.06 (3H, s, H-13 or H-14), 0.83 (3H, s, H-13 or H-14).

¹³C NMR (175 MHz, CDCl3) δ 172.2 (C-2), 170.2 (C-5′), 150.8 (C-6′), 140.7 (C-3′), 138.1 (C-7), 136.6 (C-12), 134.7 (C-4′), 109.9 (C-3), 100.4 (C-2′), 71.0 (C-5), 70.7 (C-6), 43.4 (C-4), 40.2 (C-9), 34.9 (C-8), 34.8 (C-11), 29.9 (C-15), 29.2 (C-13 or C-14), 29.1 (C-13 or C-14), 21.7 (C-7′), 19.5 (C-10).

IR (film) νmax 3479, 2930, 2861, 1785, 1754, 1682, 1457, 1346, 1191, 1085, 1023, 958 cm⁻¹.

HRESIMS m/z 363.1808 [M+H]⁺ (calc. for C20H27O6, 363.1807).

### (±)-SdL628 F2

### Method 1

To a solution of (±)-SdL781 F2 (19.2 mg, 0.044 mmol) in CH₃CN (0.3 mL) and water (3 drops), a solution of Sc(OTf)₃ (0.1 mg, 22 μmol, 0.5 mol %) in CH₃CN (0.3 mL) was added. The organic phase was extracted with CH₂Cl₂ (3×2 mL), washed with brine (2×3 mL) and dried over Na₂SO4. The solvents were removed and the crude product was purified by PTLC (petroleum ether/EtOAc, 60:40) to obtain the product (±)-SdL628 F2 of Formula 27 (3.8 mg, 19%) in the form of colorless oil.

### Method 2

To a solution of (4R*, 6R*)-B48 F2 (7.0 mg, 0.02 mmol) in anhydrous THF (1 mL) under argon, a solution of 3HF·NET₃ (20 μL, 0.18 mmol, 10.0 equiv.) was added. The mixture was stirred for one night at 50° C. The organic phase was cooled with a saturated aqueous solution of NaHCO₃ (1 mL) and extracted with EtOAc (3×2 mL). The organic phase was dried over Na₂SO₄ and the solvents were removed. The crude product was purified by PTLC (heptane/EtOAc, 60:40) to obtain the product (±)-SdL628 F2 of Formula 27 (6.3 mg, quantitative) in the form of colorless oil.

### (±)-SdL628 F2

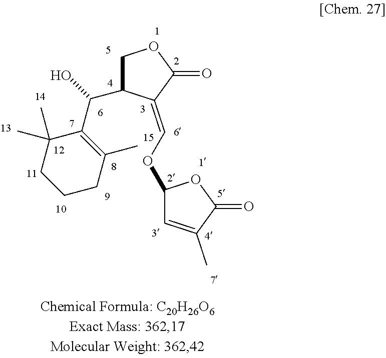

¹H NMR (500 MHz, CDCl3) δ 7.46 (1H, s, H-6′), 6.87 (1H, s, H-3′), 6.04 (1H, s, H-2′), 4.59 (1H, d, J=10.5 Hz, H-6), 4.22 (1H, dd, J=8.5, 6.5 Hz, H-5a), 4.16 (1H, d, J=10.5 Hz, H-5b), 3.86-3.82 (1H, m, H-4), 1.89-1.82 (1H, m, H-9a), 1.79 (3H, s, H-7′), 1.59-1.54 (1H, m, H-9b), 1.41-1.36 (2H, m, H-10), 1.31-1.25 (2H, m, H-11), 1.23 (3H, s, H-15), 1.03 (3H, s, H-13 or H-14), 0.83 (3H, s, H-13 or H-14)

¹³C NMR (175 MHz, CDCl₃) δ 172.2 (C-2), 170.2 (C-5′), 152.1 (C-6′), 140.7 (C-3′), 138.0 (C-7), 136.1 (C-12), 134.7 (C-4′), 109.3 (C-3), 100.9 (C-2′), 71.1 (C-5), 70.7 (C-6), 43.3 (C-4), 39.9 (C-9), 34.8 (C-8), 34.5 (C-11), 29.9 (C-15), 29.2 (C-13 or C-14), 28.9 (C-13 or C-14), 21.6 (C-7′), 19.1 (C-10).

IR (film) νₘₐₓ 3479, 2961, 2925, 2861, 1783, 1750, 1682, 1345, 1260, 1189, 1089, 1022, 955, 801 cm⁻¹.

HRESIMS m/z 363.1793 [M+H]⁺ (calc. for C₂₀H₂₇O₆, 363.1807).

(±)-SdL628 F1 and (±)-SdL628 F2 are alcohol-type cannalactone analogs corresponding to general Formula 5.

### Example 6: Assessment of Biological Activity

The biological evaluation of the cannalactone analogs according to the invention synthesized in Examples 2 (aromatic type analogs SdL625 F1+SdL625 F2), 3 (diene type analogs SdL646 F1+SdL646 F2), 4 (silylated type analogs SdL781F1+SdL781F2) and 5 (alcohol type analogs SdL628 F1+SdL628 F2) was tested for one of the activities that these analogs could ensure in hemp: the germination of a parasitic plant, P. ramosa.

The results were compared with those obtained on a reference synthetic analog, (±)-GR24 [17], and natural (+)-cannalactone [1], [4] isolated from hemp exudates:

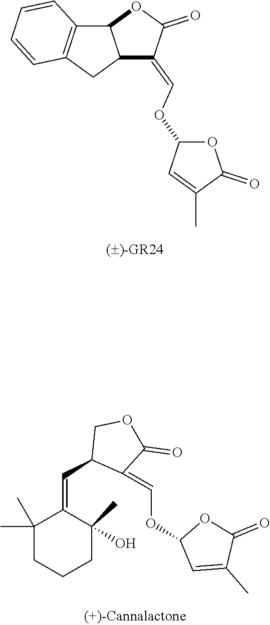

In particular, the germination stimulation activity of parasitic plant seeds was evaluated on two populations of P. ramosa. P. ramosa 1, taken from rapeseed while P. ramosa 2a is harvested from a hemp parcel [2]-[4].

### Protocol

The protocol used, developed by Pouvreau et al. [19], allows testing the biological activity of molecules or biological extracts on the germination of parasitic plant seeds in 96-well plates routinely (as shown in [FIG. 3]). This technique dispenses with the germinated seed counting that was previously used.

The cannalactone analogs synthesized in Examples 2 to 5 were tested by this protocol and their maximum activity (Maximum Germination) (as shown in [FIG. 4]) as well as the median effective concentration (EC₅₀) (as shown in [FIG. 6]) were modeled from the dose-response curve (as shown in [FIG. 5]). This measurement represents the effective concentration that induces a median response between the baseline and the maximum germination effect. [FIG. 5] shows that the analogs of Examples 2 to 5 all appear to demonstrate maximum germination capacity on the two type 2a populations of the same order as (±)-GR24. Their maximum germination capacity on type 1 seems reduced, the trend observed is closer to that obtained for (+)-cannalactone.

All the analogs synthesized in Examples 2 to 5 have EC₅₀ lower than 10⁻⁸ M for both types of populations ([FIG. 6]). Some molecules such as (±)-SdL628 F1 and (±)-SdL781 F2 have a biological activity on P. ramosa 2a at a lower concentration, therefore higher than that of the natural molecule

$$( {Ec}_{50} [ ( \pm ) - SdL 628 F 1 ] = 9.6 \times {10}^{- 12} M versus {EC}_{50} [ ( + ) - cannalactone ] = 1. \times {10}^{- 10}$$

M). The specificity of these analogs is also accentuated (as shown in [FIG. 7]), r_{EC50} [(±)-SdL628 F1]˜100 versus r_{EC50} [(+)-SdL19]˜10, and approaches that of natural cannalactone.

### BIBLIOGRAPHIC REFERENCES

1. Hamzaoui, O. et al., Proceedings of the 15^{th} World Congress on Parasitic Plants; Amsterdam, The Netherlands (2019): 32.
2. Stojanova, B., Delourme, R., Duffé, P., Delavault, P. & Simier, P. Genetic differentiation and host preference reveal non-exclusive host races in the generalist parasitic weed Phelipanche ramosa. Weed Res. 59, 107-118, doi:10.1111/wre.12353 (2019).
3. Huet, S., Pouvreau, J.-B., Delage, E., Delgrange, S., Marais, C., Bahut, M., Delavault, P., Simier, P. & Poulin, L. Populations of the Parasitic Plant Phelipanche ramosa Influence Their Seed Microbiota. Front. Plant Sci. 11, 1075, doi:10.3389/fpls.2020.01075 (2020).
4. Daignan Fornier, S., de Saint Germain, A., Retailleau, P., Pillot, J.-P., Taulera, Q., Andna, L., Miesch, L., Rochange, S., Pouvreau, J.-B. & Boyer, F.-D. Noncanonical Strigolactone Analogues Highlight Selectivity for Stimulating Germination in Two Phelipanche ramosa Populations. J. Nat. Prod 85, 1976-1992, doi:10.1021/acs.jnatprod.2c00282 (2022).
5. Delavault, P., Montiel, G., Brun, G., Pouvreau, J. B., Thoiron, S. & Simier, P. Communication Between Host Plants and Parasitic Plants. Adv. Bot. Res. 82, 55-82, doi:10.1016/bs.abr.2016.10.006 (2017).
6. Xie, X., Yoneyama, K. & Yoneyama, K. The Strigolactone Story. Annu. Rev. Phytopathol. 48, 93-117, doi:10.1146/annurev-phyto-073009-114453 (2010).
7. Daignan-Fornier, S.; Keita, A.; Boyer, F.-D., Chemistry of Strigolactones, Key Players in Plant Communication. ChemBioChem, n/a, (n/a), doi:10.1002/cbic.202400133 (2024).
8. Cook, C. E., Whichard, L. P., Turner, B. & Wall, M. E. Germination of Witchweed (Striga lutea Lour)—Isolation and Properties of a Potent Stimulant. Science 154, 1189-1190, doi:10.1126/science.154.3753.1189 (1966).
9. Akiyama, K., Matsuzaki, K. & Hayashi, H. Plant sesquiterpenes induce hyphal branching in arbuscular mycorrhizal fungi. Nature 435, 824-827, doi:10.1038/nature03608 (2005).
10. Gomez-Roldan, V., Fermas, S., Brewer, P. B., Puech-Pages, V., Dun, E. A., Pillot, J.-P., Letisse, F., Matusova, R., Danoun, S., Portais, J.-C., Bouwmeester, H., Bécard, G., Beveridge, C. A., Rameau, C. & Rochange, S. F. Strigolactone inhibition of shoot branching. Nature 455, 189-194, doi:10.1038/nature07271 (2008).
11. Umehara, M., Hanada, A., Yoshida, S., Akiyama, K., Arite, T., TakedaKamiya, N., Magome, H., Kamiya, Y., Shirasu, K., Yoneyama, K., Kyozuka, J. & Yamaguchi, S. Inhibition of shoot branching by new terpenoid plant hormones. Nature 455, 195-200, doi:10.1038/nature07272 (2008).
12. Lopez-Obando, M., Ligerot, Y., Bonhomme, S., Boyer, F.-D. & Rameau, C. Strigolactone biosynthesis and signaling in plant development. Development 142, 3615-3619, doi:10.1242/dev.120006 (2015).
13. Boyer, F.-D., de Saint Germain, A., Pillot, J. P., Pouvreau, J.-B., Chen, V. X., Ramos, S., Stevenin, A., Simier, P., Delavault, P., Beau, J.-M. & Rameau, C. Structure-activity relationship studies of strigolactone-related molecules for branching inhibition in garden pea: molecule design for shoot branching. Plant Physiol. 159, 1524-1544, doi:10.1104/p.p.112.195826 (2012).
14. Boyer, F.-D., de Saint Germain, A., Pouvreau, J.-B., Clavé, G., Pillot, J.-P., Roux, A., Rasmussen, A., Depuydt, S., Lauressergues, D., Frei dit Frey, N., Heugebaert, T. S. A., Stevens, C. V., Geelen, D., Goormachtig, S. & Rameau, C. New Strigolactone Analogs as Plant Hormones with Low Activities in the Rhizosphere. Mol. Plant 7, 675-690, doi:10.1093/mp/sst163 (2014).
15. Jas, G. Ein einfacher Zugang zu 4-Brom-2-(tert-butyldimethylsiloxy)furan aus Tetrahydro-2,4-dioxofuran. Synthesis 1991, 965-966, doi:10.1055/s-1991-26618 (1991).
16. Macalpine, G. A.; Raphael, R. A.; Shaw, A.; Taylor, A. W.; Wild, H. J. Synthesis of Germination Stimulant (±)-Strigol. J. Chem. Soc., Perkin Trans. 1 1976, (4), 410-416. DOI: 10.1039/P19760000410.
17. de Saint Germain, A., Retailleau, P., Norsikian, S., Servajean, V., Pelissier, F., Steinmetz, V., Pillot, J.-P., Rochange, S., Pouvreau, J.-B. & Boyer, F. D. Contalactone, a contaminant formed during chemical synthesis of the strigolactone reference GR24 is also a strigolactone mimic. Phytochemistry 168, 112112, doi:10.1016/j.phytochem.2019.112112 (2019).
18. Johnson, A. W., Gowda, G., Hassanali, A., Knox, J., Monaco, S., Razavi, Z. & Rosebery, G. The Preparation of Synthetic Analogs of Strigol. J. Chem. Soc., Perkin Trans. 1, 1734-1743, doi:10.1039/P19810001734 (1981).
19. Pouvreau, J.-B.; Gaudin, Z.; Auger, B.; Lechat, M. M.; Gauthier, M.; Delavault, P.; Simier, P. A high-throughput seed germination assay for root parasitic plants. Plant Methods 9 (1), 32. doi: 10.1186/1746-4811-9-32 (2013).

1. A cannalactone analog, wherein the cannalactone corresponds to the general Formula (1): 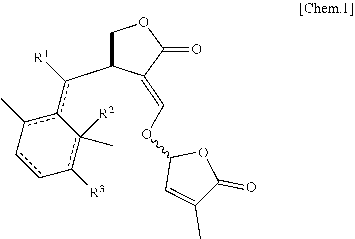
wherein:
R¹ denotes the hydrogen atom H, the hydroxyl group OH or the OSiR⁴₃ group,
R² and R³ each denote hydrogen atom H or methyl radical CH₃,
R⁴ denotes an alkyl group, and
the 6-membered carbon ring which may be aromatic or of the cyclohexene or cyclohexane type.

2. The cannalactone analog according to claim 1, wherein the cannalactone analog is aromatic of “cis” and “trans” stereochemistry and corresponds to Formula (2): 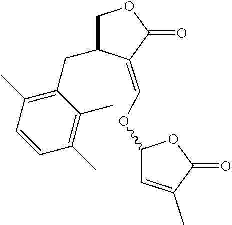
wherein:
R¹, R² and R³ denote the hydrogen atom H, and—the 6-membered carbon ring is aromatic.

3. The cannalactone analog according to claim 1, wherein the cannalactone analog is of the “cis” and “trans” stereochemistry diene type and corresponds to Formula (3): 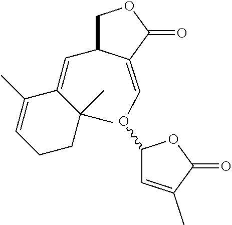
wherein:
R¹ and R³ denote the hydrogen atom H,
R² denotes the methyl group, and
the 6-membered carbon ring is of the cyclohexene type.

4. The cannalactone analog according to claim 1, wherein the cannalactone analog is of the “cis” and “trans” stereochemistry sillilate type and corresponds to Formula (4): 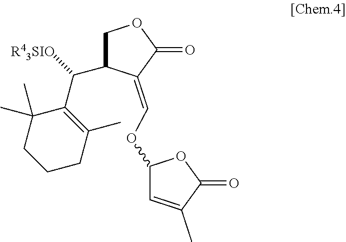
wherein:
R¹ denotes the OSiR⁴₃ group,
R² denotes the methyl group, —R³ denotes hydrogen atom H, and
the 6-membered carbon ring is of the cyclohexene type.

5. The cannalactone analog according to claim 1, wherein the cannalactone analog is of the “cis” and “trans” stereochemistry alcohol type and corresponds to Formula (5): 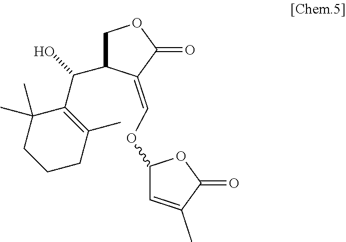
wherein:
R¹ denotes the hydroxyl group OH, and
R² denotes the methyl group, —R³ denotes hydrogen atom H, and
the 6-membered carbon ring is of the cyclohexene type.

6. A method for synthesizing a cannalactone analog as defined according to claim 2, the method comprising:
a reaction A) of coupling commercial β-cyclocitral with unbromofuran C4 of Formula (6): 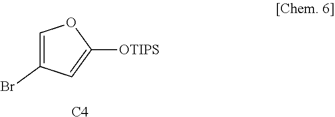
to obtain an alcohol B20 of Formula (7): 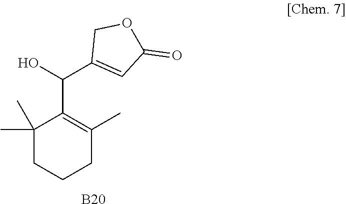
a step B) of reducing the alcohol B20 of Formula (7), to obtain a mixture of diastereoisomers of the allyl alcohol, followed by a step of separating said diastereoisomers to retain the diastereoisomer (4R*, 6R*)-B21 of Formula (8): 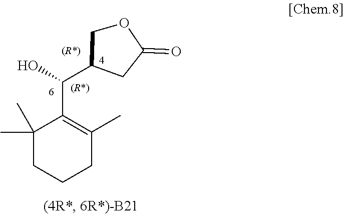
a step C2) of epoxidizing the diastereoisomer (4R*,6R*)-B21 of Formula (8) to obtain an epoxy alcohol B22 of Formula (9): 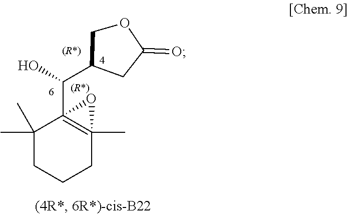
a step D2) of dehydrating and rearranging the epoxyalcohol B22 of Formula (9) to obtain a benzyl compound B38 of Formula (10): 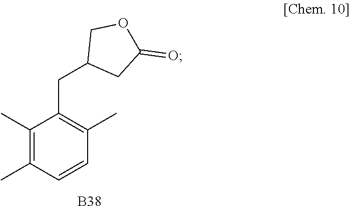 [SMILES: "B38" ; Cc1ccc(C)c(CC2COC(=O)C2)c1C]
a step E2) of formylation in basic medium of the benzyl compound B38 of Formula (10) to obtain an enol B40 of Formula (11): 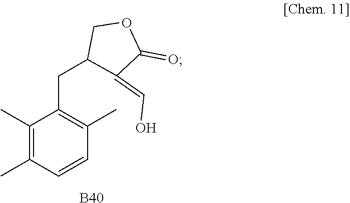 [SMILES: "B40" ; Cc1ccc(C)c(CC2COC(=O)/C2=C/O)c1C]
a step F2) of O-alkylation of enol B40 to obtain the analog of Formula (2).

7. The method for synthesizing a cannalactone analog as defined according to claim 3, comprising:
performing (a) a reaction A) of coupling commercial β-cyclocitral with unbromofuran C4 of Formula (6): 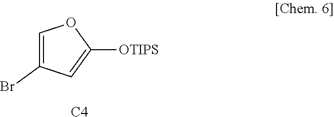
to obtain an alcohol B20 of Formula (7): 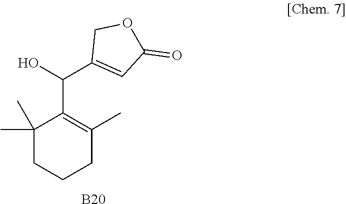
and (b) a step B) of reducing the alcohol B20 of Formula (7), to obtain a mixture of diastereoisomers of the allyl alcohol, followed by a step of separating said diastereoisomers to retain the diastereoisomer (4R*, 6R*)-B21 of Formula (8): 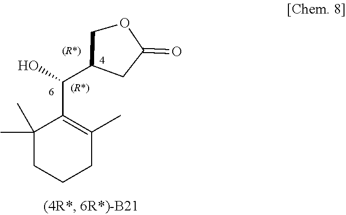
wherein (a) and (b) are followed by
a step C3) of mesylating the diastereoisomer (4R*, 6R*)-B21 of Formula (8) to obtain after dehydration and rearrangement the diene (E)-B25 of Formula (13): 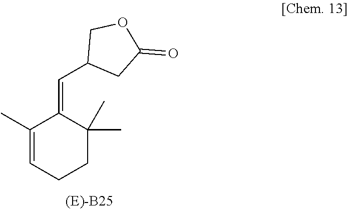 [SMILES: CC1=CCCC(C)(C)/C1=C\C1COC(=O)C1]
a step E3) of formylation in basic medium of diene (E)-B25 of Formula 13 to obtain an enol B42 of Formula (14): [Chem. 14]: 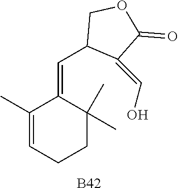 [SMILES: "B42" ; CC1=CCCC(C)(C)/C1=C\C1COC(=O)/C1=C/O]
a step F3) of O-alkylation of enol B42 to obtain the analog of Formula (3).

8. A method for synthesizing a cannalactone analog as defined according to claim 4, characterized in that it comprises the following comprising:
performing (a) a reaction A) of coupling commercial β-cyclocitral with unbromofuran C4 of Formula (6): 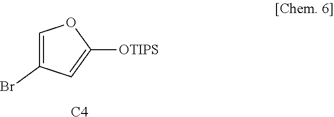
to obtain an alcohol B20 of Formula (7): 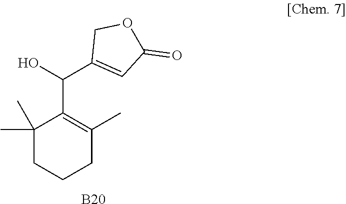
and (b) a step B) of reducing the alcohol B20 of Formula (7), to obtain a mixture of diastereoisomers of the allyl alcohol, followed by a step of separating said diastereoisomers to retain the diastereoisomer (4R*, 6R*)-B21 of Formula (8): 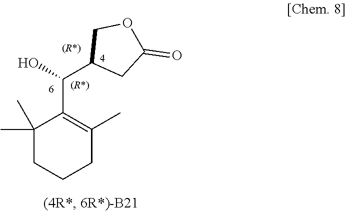
wherein (a) and (b) are followed by
a step C4) of protecting the diastereoisomer (4R*, 6R*)-B21 of Formula 8 to obtain the protected compound (4R*, 6R*)-B45 of Formula (15): [Chem. 15]: 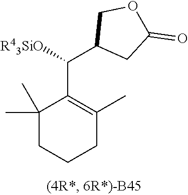
a step E4) of formylation in basic medium of the protected compound of Formula 16 to obtain an enol (4R*, 6R*)-B46 of Formula (16): 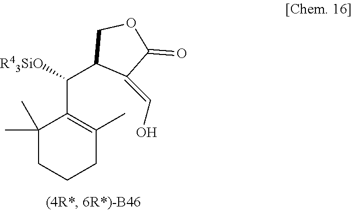
a step F4) of O-alkylation of enol B46 to obtain the analog of Formula (4).

9. A method for synthesizing a cannalactone analog, comprising:
forming the cannalactone analog according to the method as defined in claim 8, followed by
a step G5) of deprotecting and separating the diastereoisomers of the analog of Formula (4), to obtain the analog of Formula (5) [Chem.5]

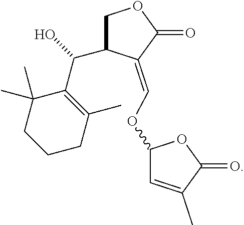

10. A method comprising utilizing a cannalactone analog as defined according to claim 1 as a parasitic plant seed germination stimulant.

11. The method according to claim 10, wherein the parasitic plant seed germination stimulant is a germination stimulant of P. ramosa 1 and P. ramosa 2a seeds.

12. The method according to claim 10, wherein the cannalactone analog is utilized for the suicidal germination of parasitic plants of the Striga, Orobanche and Phelipanche type.
